(19) United States
(12) Patent Application Publication
(10) Pub. No.: US 2025/0359854 A1
(43) Pub. Date: Nov. 27, 2025

(54) ULTRASOUND DIAGNOSTIC APPARATUS, ULTRASOUND DIAGNOSTIC PROGRAM, AND ULTRASOUND DIAGNOSTIC METHOD

(71) Applicant: KONICA MINOLTA, INC., Tokyo (JP)

(72) Inventor: Kazuya Takagi, Tokyo (JP)

(73) Assignee: KONICA MINOLTA, INC., Tokyo (JP)

(21) Appl. No.: 19/219,130

(22) Filed: May 27, 2025

(30) Foreign Application Priority Data
May 27, 2024 (JP) 2024-085753

(51) Int. Cl. A61B 8/00; A61B 8/06; A61B 8/08

(52) U.S. Cl. CPC A61B 8/54; A61B 8/06; A61B 8/0891; A61B 8/463; A61B 8/488

(57) ABSTRACT

An ultrasound diagnostic apparatus, which displays a tissue other than an annotated blood vessel region with high visibility when performing annotation-display of the blood vessel region using an AI technology, has a prevention function of preventing puncture from being performed in a state in which information indicating a position of a blood vessel region recognized using a learned model is displayed on a display screen, and includes a display controller that displays a warning for preventing puncture while displaying the information indicating the position of the blood vessel region on the display screen.

### REFERENCE TO RELATED APPLICATIONS

The present invention relates to and asserts priority from Japanese patent application No. 2024-085753 filed on May 27, 2024, and incorporates the entirety of the contents and subject matter of all the above application herein by reference.

### BACKGROUND OF THE INVENTION

### Technical Field

The present invention relates to an ultrasound diagnostic apparatus, an ultrasound diagnostic program, and an ultrasound diagnostic method.

### Description of Related Art

Conventionally, many medical diagnoses based on ultrasound images of organs and tissues of living bodies have been performed. An ultrasound diagnostic apparatus that generates the ultrasound image transmits ultrasound waves to a living body, receives ultrasound waves (echoes) reflected on organs and tissues, and images them as a cross-unital image.

Furthermore, in recent years, a technology has been known that performs annotation-display of a blood vessel region depicted on an ultrasound image using an AI technology.

For example, as disclosed in the patent literature, Japanese Unexamined Patent Publication No. 2023-026610, a technology is known in which an ultrasound image generated by an ultrasound diagnostic apparatus is analyzed so that an identified blood vessel region is displayed with highlight. Specifically, the technology disclosed in the above patent literature stores typical pattern data of a blood vessel region in advance as a template and calculates a degree of similarity with respect to the pattern data while searching the ultrasound image with the template. Then, the technology regards a place where the degree of similarity is equal to or more than a threshold value and maximum as a place where a blood vessel region exists. Furthermore, the above patent literature describes that the degree of similarity can be calculated with a general image recognition method or the like using machine learning or deep learning in addition to the template matching.

In the present specification, techniques such as machine learning and deep learning for identifying a blood vessel region are collectively referred to as artificial intelligence (AI).

### SUMMARY OF THE INVENTION

### Technical Problems

The above-described related art allows easy grasping of a blood vessel region, and thus the related art is useful when performing a procedure of inserting an insertion object such as a puncture needle and a catheter into a blood vessel of a subject, so-called puncture. However, a blood vessel region may be sometimes erroneously recognized using the AI technology.

When an annotation-display is performed by superimposing a predetermined color on a blood vessel detected using the AI technology, a tissue other than a blood vessel region located in the vicinity of the blood vessel region, for example, there may be a case in which annotation-display of a nerve region is also performed. There may be a case in which it is difficult to visually recognize a tissue other than the annotated blood vessel region located in the vicinity of the blood vessel region. Therefore, for example, when a health care professional punctures a patient while viewing an ultrasound image, there is a risk that a nerve region of the patient may also be injured.

An object of the present invention is to provide an ultrasound diagnostic apparatus, an ultrasound diagnostic program, and an ultrasound diagnostic method capable of displaying a tissue other than an annotated blood vessel region with high visibility when performing an annotation-display of the blood vessel region using the AI technique.

### Solution to Problem

The above-described object is solved by the following solutions according to one aspect of the present invention.

An ultrasound diagnostic apparatus implementing a prevention function of preventing puncture from being performed in a state in which information indicating a position of a blood vessel region recognized using a learned model is displayed on a display screen.

### BRIEF DESCRIPTION OF THE DRAWINGS

Hereinbelow, embodiments of the present invention is described in detail with reference to the drawings. The advantageous effect and features provided by one or more embodiments of the present invention become more fully understood from the detailed description given hereinbelow and the appended drawings, which are given by way of illustration only and thus are not intended as a definition of the limitations of the present invention.

FIG. 1 is a block diagram illustrating a configuration of an ultrasound diagnostic apparatus of an embodiment of the present invention.

FIG. 2 is a view illustrating a display example of a display unit.

FIG. 3 is a view illustrating a display example in a case in which information indicating a position of a blood vessel region is superimposed and displayed.

FIG. 4 is a flowchart illustrating an operation in a case in which information indicating a position of a blood vessel region is superimposed and displayed.

FIG. 5 is a view illustrating a display example in a case in which a user taps an operation instruction button for a puncture mode when information indicating a position of a blood vessel region is superimposed and displayed on a B-mode image.

FIG. 6 is a flowchart of processing for hiding information indicating the position of the blood vessel region in response to detection of tapping on an operation instruction button in a puncture mode.

FIG. 7 is a view showing a display portion in which the operation instruction button of a blood vessel display mode is inactivated.

FIG. 8 is a flowchart for explaining display processing of FIG. 7 by the ultrasound diagnostic apparatus.

FIG. 9 is a diagram showing an example which, in the puncture mode, displays side by side on the same screen a B-mode image in which a puncture needle is highlighted and a B-mode image in which information indicating the position of the blood vessel region is superimposed to be displayed.

FIG. 10 is a flowchart for explaining the display process of the ultrasound diagnostic apparatus in FIG. 9.

FIG. 11 is a diagram illustrating a display example when a blood vessel display mode is operated while displaying a Doppler display mode.

FIG. 12 is a flowchart explaining the display processing of the ultrasound diagnostic apparatus in FIG. 11.

FIG. 13 is a diagram illustrating another display example when the blood vessel display mode is operated while displaying the Doppler display mode.

FIG. 14 is a flowchart for explaining the display processing of the ultrasound diagnostic apparatus in FIG. 13.

FIG. 15 is a diagram for explaining a display control when there is an operation instruction issued in the Doppler display mode while displaying the blood vessel display mode.

FIG. 16 is a flowchart for explaining the display processing of the ultrasound diagnostic apparatus in FIG. 15.

FIG. 17 is a diagram illustrating another display example when there is an operation instruction in the Doppler display mode issued while displaying the blood vessel display mode.

FIG. 18 is a flowchart for explaining the display processing of the ultrasound diagnostic apparatus in FIG. 17.

### DETAILED DESCRIPTION

The ultrasound diagnostic apparatus according to the embodiment displays on a display screen by superimposing on a B mode image information indicating a position of a blood vessel region, which position is recognized from a brightness (B) mode image of a subject captured through an ultrasound echo using an AI model that is learned using machine learning or deep learning. The ultrasound diagnostic apparatus has a prevention function of preventing puncture from being performed in the above display state. This also allows tissues other than annotated blood vessel region to be displayed with high visibility when performing annotation-display of the blood vessel region using the AI technique.

Hereinbelow, the embodiments of the present invention are described in detail with reference to the drawings. However, the scope of the present invention is not limited to the disclosed embodiments.

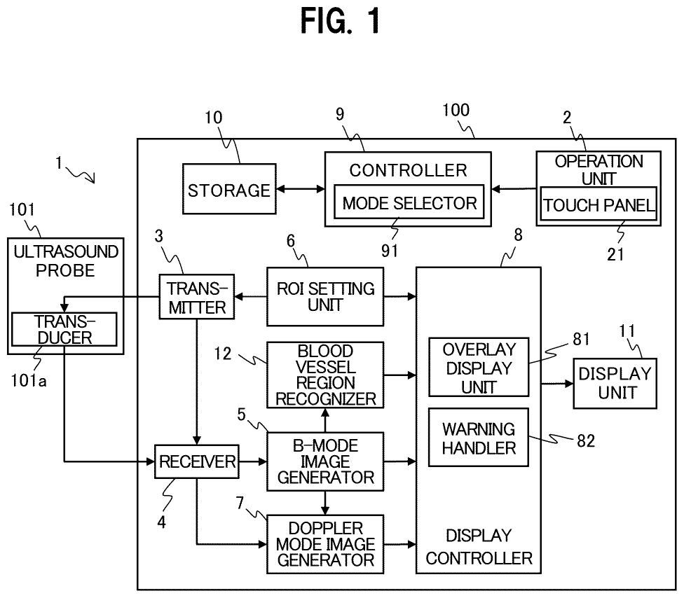

FIG. 1 is a block diagram illustrating the configuration of an ultrasound diagnostic apparatus 1 according to an embodiment.

The ultrasound diagnostic apparatus 1 is configured such that an ultrasound probe 101 is connected to a main body 100 via a cable.

The main body 100 of the ultrasound diagnostic apparatus 1 includes an operation unit 2, a transmitter 3, a receiver 4, a B-mode image generator 5, and an ROI (Region Of Interest) setting unit 6, a Doppler mode image generator 7, a display controller 8, a controller 9, a storage unit 10, a display unit 11, and a blood vessel region recognizer 12.

The ultrasound probe 101 has a plurality of transducers (piezoelectric transducer elements) 101a arranged in one-dimensional direction. Each of the transducers 101a is applied with a drive signal from the transmitter 3 described below to generate ultrasound waves, and a plurality of the ultrasound waves interfere with each other to generate an ultrasound beam. The ultrasound beam is radiated to a subject. Next, the ultrasound probe 101 receives reflected waves (echoes) of the ultrasound waves from the inside of the subject through the plurality of transducers 101a, converts the received waves into electrical signals, and outputs the electrical signals to the receiver 4 described below.

The operation unit 2 is a display instructor that detects an operation instruction from a user to notify the controller 9 of a mode command based on the operation instruction from the user, and is, for example, a touch panel 21. For example, the operation unit 2 notifies, based on the operation instruction from the user, the controller 9 on which mode is to be executed, a mode in which a B-mode image is displayed or a mode in which a Doppler mode image is superimposed and displayed on the B-mode image. The B-mode image is an ultrasound image in which an amplitude of a reflected wave (echo) of an ultrasound wave is represented by brightness (luminance), and the Doppler mode image is a color flow mode image.

The operation unit 2 also includes a function of receiving a designation input of a position of the ROI on which a user displays the Doppler mode image on the B-mode image.

Here, it is assumed that display modes for the Doppler mode image include a V mode in which a flow velocity and a direction of blood flow are displayed in color by a blood flow velocity V as a blood flow signal indicating a state of the blood flow, a P mode in which the power of the blood flow is displayed in color by a power P of the blood flow as the blood flow signal, and a V-T mode in which the flow velocity and dispersion of the blood flow are displayed in color by the blood flow velocity V and the dispersion T as the blood flow signal.

When detecting a user's operation instruction for Doppler mode image display, the operation unit 2 further detects the above-described display mode instruction. The display modes for the Doppler mode image display may include a T (dispersion) mode, a dP (directed power) mode, and the like. Thus, the display modes of the Doppler mode image display include the color Doppler mode (the V mode, the V-T mode, and the like) and the power Doppler mode (the P mode and the like).

Further, the operation unit 2 detects a user operation instruction of a puncture mode in which the puncture needle that is inserted with assistance of a puncture adapter or a puncture guide is highlighted in the B-mode image, and notifies the controller 9 of a mode command.

In addition, the operation unit 2 detects a user operation instruction of a blood vessel display mode in which information indicating a position of a blood vessel region recognized from a B-mode image of an ultrasound echo of the subject by using an AI model which has been learned through of machine learning or deep learning is superimposed and displayed on a display screen of the B-mode image. Then, the operation unit 2 notifies the controller 9 of a blood vessel display mode command as a user operation instruction.

More specifically, the display controller 8 displays an instruction button on a display unit 11 which displays a B-mode image, or the like described below. The operation unit 2 detects tapping on an instruction button through the touch panel 21, and regards the tapping as a user's operation instruction. Note that the detection of the user's operation instruction is not limited to the touch panel 21 but may be performed by other detection means.

The transmitter 3 transmits a high-voltage drive signal at a predetermined timing to the transducer 101a of the ultrasound probe 101 to irradiate the subject with an ultrasound beam.

When displaying a color Doppler image, the transmitter 3 performs transmission processing for the Doppler mode for displaying a blood flow image in addition to transmission processing for displaying a B-mode image under control of the controller 9. At this time, for example, the transmitter 3 alternately performs a B-mode scanning and a Doppler mode scanning.

In the Doppler mode scanning, while an ultrasound beam is repeatedly transmitted n times in the same directions (same line), an ultrasound beam is transmitted in all directions (all lines) of the ROI set by an ROI setting unit 6. The number of repetitions n is a dozen times, for example, 16, and referred to as the number of ensembles. Further, the transmitter 3 designates additional information of the transmission processing for the B-mode image or the Doppler mode image at the time of the transmission processing to supply the additional information to the receiver 4.

The receiver 4 amplifies analog electrical signals converted from the reflected waves (echoes) of the ultrasound waves received by the ultrasound probe 101, performs A/D conversion, and performs phasing addition, thereby generates digital reception signals.

In addition, the receiver 4 acquires additional information from the transmitter 3, and supplies a reception signal to the B-mode image generator 5 if the acquired additional information is additional information for a B-mode image. If the acquired additional information is additional information for a Doppler mode image, the reception signal is supplied to the Doppler mode image generator 7. Hereinbelow, a reception signal for generating a B-mode image is referred to as a “B-mode reception signal”, and a reception signal for generating a Doppler mode image is referred to as a “Doppler mode reception signal”.

Under the control of the controller 9, the B-mode image generator 5 performs envelope detection, logarithmic compression, and the like on the B-mode reception signals input from the receiver 4, adjusts the dynamic range and the gain to convert luminance, and thereby generates B-mode image data to output it to the display controller 8.

Under the control of the controller 9, the ROI setting unit 6 sets a region of interest (ROI) of the Doppler mode image in the transmitter 3 and the display controller 8 according to the ROI designation information input by the user via the operation unit 2.

Under the control of the controller 9, the Doppler mode image generator 7 generates Doppler mode image data in accordance with the Doppler mode reception signals input from the receiver 4 and outputs the Doppler mode image data to the display controller 8.

The display controller 8 performs processing of superimposing the Doppler mode image data and information indicating the position of the blood vessel region on the B-mode image data to display the superimposed data on the display unit 11.

The display controller 8 displays the B-mode image generated by the B-mode image generator 5 on the display unit 11 in a display mode of the B-mode image.

Furthermore, in a display mode of the Doppler mode image, the display controller 8 has an overlay display controller 81 superimpose the Doppler mode image generated by the Doppler mode image generator 7 at the position of the selected ROI on the B-mode image generated by the B-mode image generator 5, and displays the superimposed data on the display unit 11.

The warning handler 82 is a processing unit for displaying warning information on the display unit 11 in order to prevent puncture from being performed in a state in which the information indicating the position of the blood vessel region is displayed being superimposed on the display screen of the B-mode image. Details is described below.

In addition to the display of the warning information by the warning handler 82, for example, a warning regarding puncture may be notified by voice or the like, which is not limited thereto.

The controller 9 sets operation content of each processing unit of the main body 100 and controls the entire operation of the main body 100. In particular, a mode selector 91 of the controller 9 controls the display controller 8 to display setting buttons for a display mode and an operation type on the display unit 11. For example, the setting buttons include buttons for setting a Doppler image display mode for detecting a blood flow signal using the Doppler mode and generating and displaying a Doppler image, and a puncture mode setting button for assisting puncture by highlighting a puncture needle in a B-mode image. Further, the setting buttons include a setting button for a blood vessel region display mode in which information indicating the position of the blood vessel region is overlaid and displayed on the B-mode image.

Further, the operation unit 2 detects tapping of the setting button displayed on the display unit 11 by the user through the touch panel 21 provided on the display unit 11 and notifies the mode selector 91 of the user's operation instruction corresponding to the setting button. The mode selector 91 sets the operation content of each processing unit according to the notified operation instruction from the user such as the Doppler image display, the puncture mode, or the blood vessel region display mode.

In the above description, the operation unit 2 detects that the user taps the setting button of the display mode, or the operation type displayed on the display unit 11. However, a physical operation switch corresponding to the setting button displayed on the display unit 11 may be provided, so that the operation unit 2 may detect the switch operation of the user.

The storage unit 10 stores ultrasound image data, a gain matrix, a frame rate, and the like. The ultrasound image data includes the B-mode image data, the Doppler mode image data, composite image data, and the like.

The display unit 11 is a so-called monitor display such as an LCD (Liquid Crystal Display) or an EL (Electro Luminescence) display for displaying display image data output from the display controller 8. A transparent touch panel 21 is superimposed on a display surface of the display unit 11 so that tapping of a setting button by a user can be detected. Instead of the tapping of the setting button by the user, the operation instruction of the user may be performed by a mouse click or the like of the setting button.

The blood vessel region recognizer 12 is a processing unit that recognizes (determines) a blood vessel region in the B-mode image generated by the B-mode image generator 5 using, for example, a learned model obtained by causing a convolutional neural network to learn on the basis of teacher data when the blood vessel region display mode is set.

The blood vessel region recognized by the learned model is notified to the display controller 8 as information indicating the position of the blood vessel region. The information indicating the position of the blood vessel region is, for example, a contour figure of the blood vessel region or a figure painted in a predetermined color (for example, red) inside the contour, and includes position information in the B-mode image. Further, the information indicating the position of the blood vessel region may be information on coordinate positions of pixels determined as the blood vessel region in the B-mode image. The display controller 8 superimposes and displays, on the B-mode image, the information indicating the position of the blood vessel region notified to the blood vessel region recognizer 12, using an overlay display controller 81.

More specifically, the main body 100 of the ultrasound diagnostic apparatus 1 according to the embodiment is implemented by an information processing apparatus (computer) including a central processing unit (CPU) and a large-capacity recording medium such as a read only memory (ROM), a random-access memory (RAM), and a hard disk drive (HDD).

The CPU reads a diagnostic program stored in the ROM or the HDD, develops the diagnostic program in the RAM, and implements a function of each unit of the main body 100 in accordance with the developed program. The RAM forms a work area for temporarily storing various programs to be executed by the CPU and data related to these programs. The ultrasound diagnostic program may be stored and supplied in a form of a computer-readable program code in a recording medium, or may be transmitted and supplied through a network.

FIG. 1 shows an example in which the ultrasound diagnostic apparatus 1 of the embodiment includes the main body 100 provided with the display unit 11 for displaying a reflected wave (echo) of an ultrasound wave, and the ultrasound probe 101. Without being limited thereto, the ultrasound diagnostic apparatus 1 may be configured such that each processing unit of the main unit 100 may be provided as a component of an ultrasound diagnostic apparatus on a network and the ultrasound probe 101 and the display unit 11 may be connected to the ultrasound diagnostic apparatus via the network.

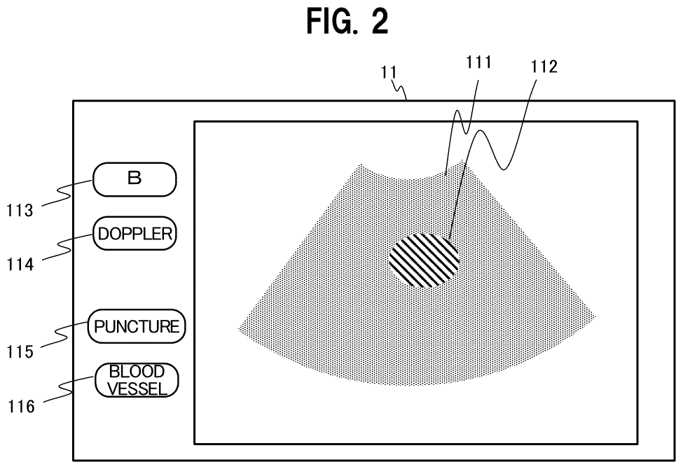

FIG. 2 is a diagram illustrating a display example of a display unit 11.

The display unit 11 in FIG. 2 displays display image data in which blood vessel region information 112 indicating the position of the blood vessel region is superimposed on the B-mode image 111. The blood vessel region information 112 is displayed as a contour line figure of the blood vessel region or a figure filled with a predetermined color inside the contour line. The predetermined color may be, for example, red.

Further, the display unit 11 displays buttons as the operation unit 2 for instructing and setting operation of the ultrasound diagnostic apparatus 1 by being tapped by the user. The B button 113 is a button for setting a display mode of a B-mode image. The Doppler button 114 is a button for setting a Doppler display mode in which a color Doppler mode image is superimposed and displayed on the B mode image.

Furthermore, the blood vessel button 116 is a button for instructing a blood vessel display mode in which information indicating a position of a blood vessel region recognized from the B-mode image is superimposed and displayed on the B-mode image. The puncture button 115 is a button for giving an instruction to display a puncture mode in which a puncture needle is highlighted. That is, the blood vessel button 116 is a display instructor for displaying information indicating the position of the blood vessel region. Note that FIG. 2 shows only buttons related to the operation of the ultrasound diagnostic apparatus 1 of the embodiment described below and omits display of the other buttons.

Next, a description is given of operation in a case in which a user taps the blood vessel button 116, which is an operation instruction button for the blood vessel display mode, and the ultrasound diagnostic apparatus 1 displays, on the B-mode image, information indicating the position of the blood vessel region recognized from the B-mode image so as to be superimposed on the ultrasound image.

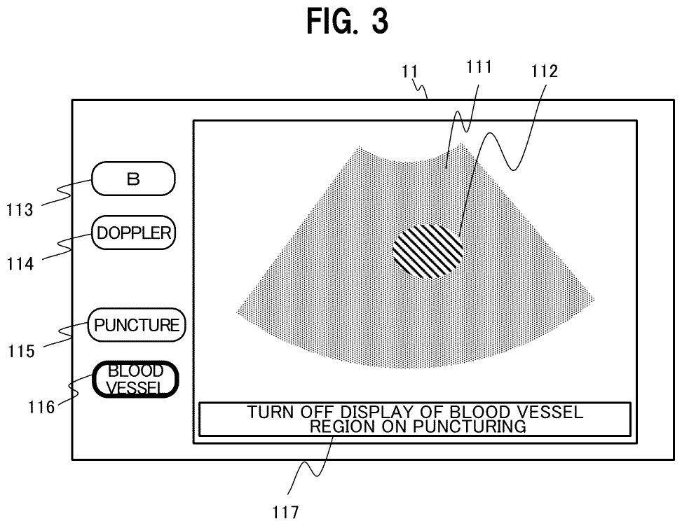

FIG. 3 is a diagram illustrating a display example in a case in which information indicating the position of a blood vessel region is superimposed and displayed.

The user taps the blood vessel button 116 instructing the blood vessel display mode, and the ultrasound diagnostic apparatus 1 detects the tapping through the touch panel 21 of the operation unit 2. This causes, as illustrated in FIG. 3, the display controller 8 to superimpose the blood vessel region information 112 on the B-mode image 111 and to display the superimposed image on the display unit 11. Furthermore, the ultrasound diagnostic apparatus 1 displays a warning message 117 for preventing puncturing while displaying the B-mode image 111. The warning regarding the puncture is, for example, “Please turn off display of blood vessel region on puncture.”

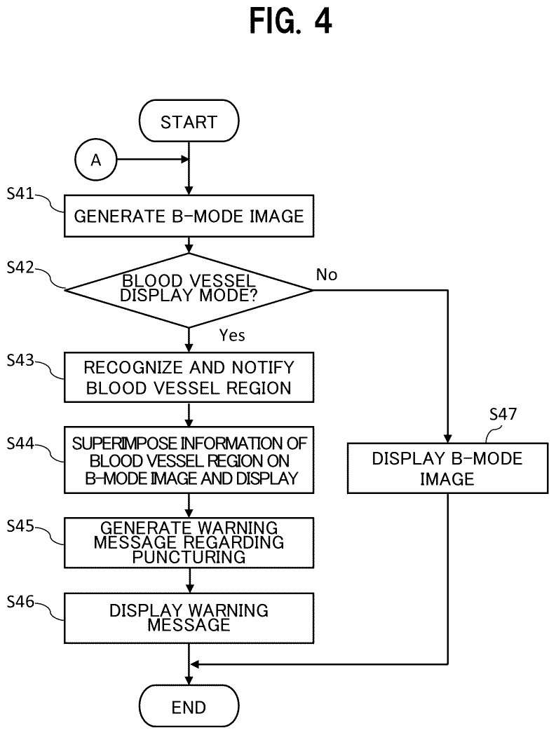

The operation of the ultrasound diagnostic apparatus 1 is described with reference to the flowchart of FIG. 4. The flowchart of FIG. 4 illustrates processing for one frame of a reflected wave (echo) of an ultrasound wave.

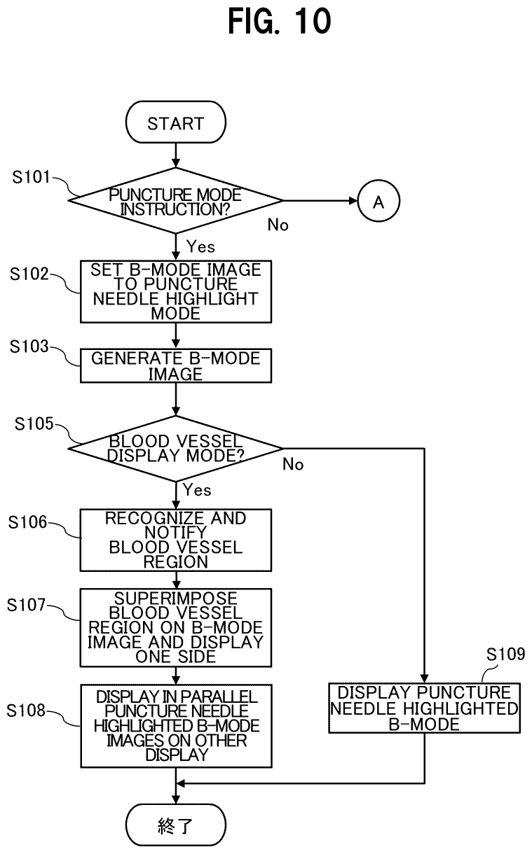

Note that FIG. 4 show an off-page connector symbol at the beginning of step S41, and that this off-page connector symbol connects to the flowchart of FIG. 10, which is described below. The same processing as that in FIG. 4 is performed in the course of the processing in FIG. 10, although details is described below.

In step S41, the B-mode image generator 5 generates a B-mode image based on reflected waves (echoes) of ultrasound waves.

In step S42, the controller 9 determines whether or not the mode is the blood vessel display mode based on detection of tapping on the blood vessels button 116 indicating the blood vessel display mode on the touch panel 21 of the operation unit 2. If the mode is the blood vessels display mode (YES in step S42), the process proceeds to step S43. If the mode is not the blood vessel display mode (NO in step S42), the process proceeds to step S47.

In step S43, the blood vessel region recognizer 12 recognizes a blood vessel region in the B-mode image generated in step S41 using the learned model and notifies the display controller 8 of information indicating the position of the recognized blood vessel region.

In step S44, the overlay display controller 81 of the display controller 8 superimposes on the B-mode image information indicating the blood vessel region recognized by the learned model, and displays the superimposed image on the display unit 11.

In step S45, the warning handler 82 of the display controller 8 generates a warning message regarding puncturing.

In step S46, the display controller 8 displays the warning message generated in step S45 on the display unit 11 and terminates the processing in FIG. 4.

In step S47, the display controller 8 displays the B-mode image generated in step S41 on the display unit 11, and terminates the processing in FIG. 4.

Next, description is given of an operation performed when the user taps the puncture button 115 instructing to display the puncture mode in which a puncture needle is highlighted in a state in which the ultrasound diagnostic apparatus 1 superimposes and displays the blood vessel region information 112 on the B-mode image 111 on the display unit 11.

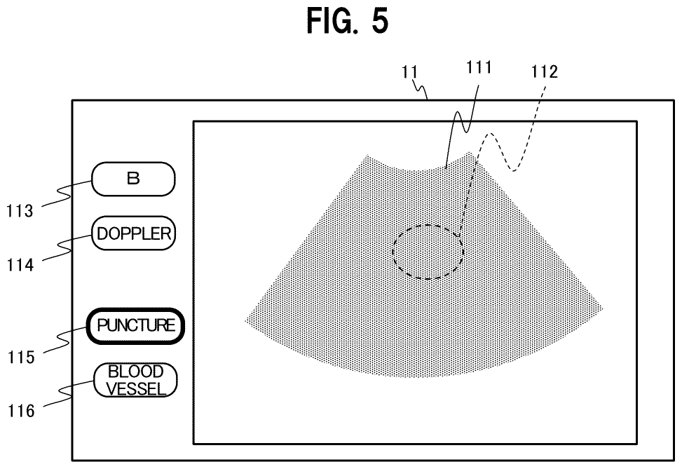

FIG. 5 is a view illustrating a display example in this case.

When detecting a user's tapping on the blood vessel button 116 that is displayed on the display unit 11 and indicates the blood vessel display mode through the touch panel 21 of the operation unit 2, the ultrasound diagnostic apparatus 1 superimposes the blood vessel region information 112 on the B-mode image 111 and displays the superimposed image on the display unit 11. Thereafter, when detecting a user's tapping on the puncture button 115 for instructing the display of the puncture mode in which the puncture needle displayed on the display unit 11 is highlighted, the ultrasound diagnostic apparatus 1 hides the blood vessel region information 112. Furthermore, the ultrasound diagnostic apparatus 1 hides the warning message regarding puncturing.

As a result, because the blood vessel region information 112 disappears, tissues other than the annotated blood vessel region can also be displayed with high visibility. That is, although there is a risk of a false recognition of the blood vessel region due to false highlighting or a degradation in the visibility of the B-mode image due to the superimposed display of the blood vessel region when the highlighting of the blood vessel is used at the time of puncturing, this risk can be avoided.

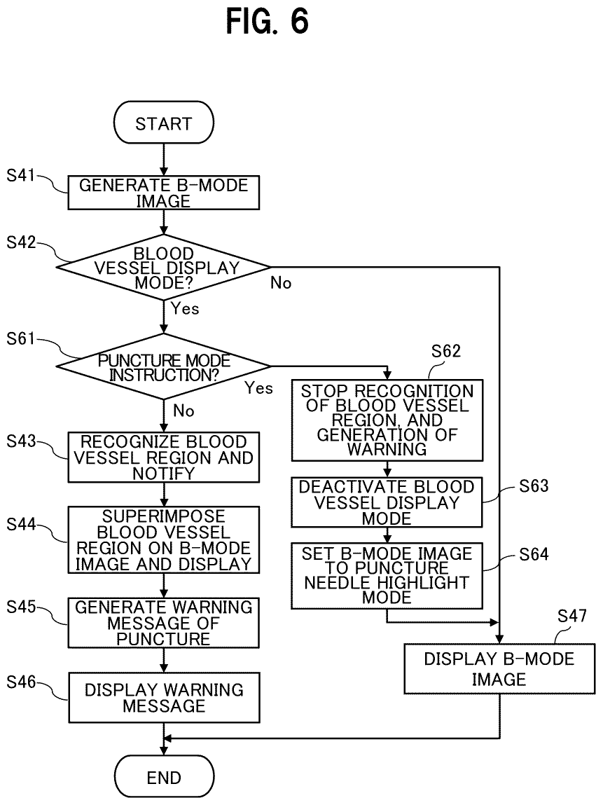

FIG. 6 is a flowchart of processing for hiding the blood vessel region information 112 in response to detection of the tapping on the puncture button 115 instructing to display the puncture mode described in FIG. 5.

The flowchart of FIG. 6 is obtained by adding processing of steps S61 to S63 to the flowchart illustrating the operation in a case in which the information indicating the position of the blood vessel region is superimposed and displayed on the B-mode image. Here, the processing of the added steps S61 to S63 is described.

In step S61, the controller 9 determines whether or not the instruction is for the puncture mode based on detection of tapping on the puncturing button 115 instructing to display the puncture mode through the mode selector 91. If the tapping is not the instruction for the puncture mode (No in step S61), the process proceeds to step S43. If the tapping is the instruction for the puncture mode (Yes in step S61), the process proceeds to step S62.

In step S62, the controller 9 controls the blood vessel region recognizer 12 to stop the recognition of the blood vessel region, and controls the warning handler 82 to stop the generation of the warning message related to puncturing.

In step S63, the display controller 8 deactivates the blood vessel button 116 for instructing the blood vessel display mode, thus invalidating the operation instruction for the blood vessel display mode.

Then, in step S64, the B-mode image generator 5 sets the B-mode image to the puncture needle highlighting mode. After step S64, the process proceeds to step S47, where the B-mode image generator 5 generates a B-mode image that is in the puncture needle highlighting mode, and the display controller 8 displays this B-mode image on the display unit 11. At this time, information indicating the blood vessel region is not displayed.

The above description explains the processing in which the controller 9 determines whether or not there is the operation instruction for the puncture mode and stops the superimposed display of the information indicating the blood vessel region. In relation to this processing, the controller 9 can perform recognition of the puncture needle in the B-mode image instead of determining the presence or absence of the operation instruction of the puncture mode by detecting the tapping of the puncture button 115 for instructing the display of the puncture mode. Then, when the puncture needle is detected, the controller 9 may stop the display of the information indicating the position of the blood vessel region. That is, when the puncture needle is detected in a state in which the information indicating the position of the blood vessel region is displayed on the display screen, the display of the information indicating the position of the blood vessel region may be stopped.

The above is the description of the display of the information indicating the position of the blood vessel region in a case in which the operation instruction for the puncture mode is provided during the display of the information indicating the blood vessel region. Next is a description of a case in which the operation instruction for the puncture mode is provided in a state in which the information indicating the blood vessel region is not displayed.

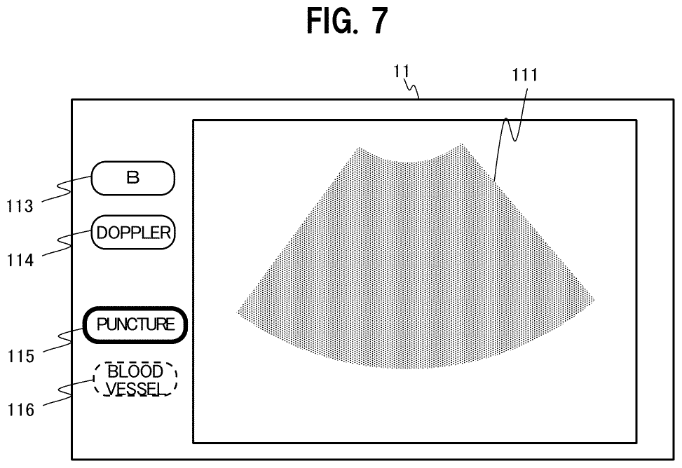

FIG. 7 is a diagram illustrating a state in which a user's tapping of the puncture button 115 instructing to display the puncture mode is performed, in a state in which only the B-mode image 111 is displayed on the display unit 11. In this case, the ultrasound diagnostic apparatus 1 inactivates the blood vessel button 116 instructing the blood vessel display mode to prohibit its selection, thus invalidating the operation instruction for the blood vessel display mode.

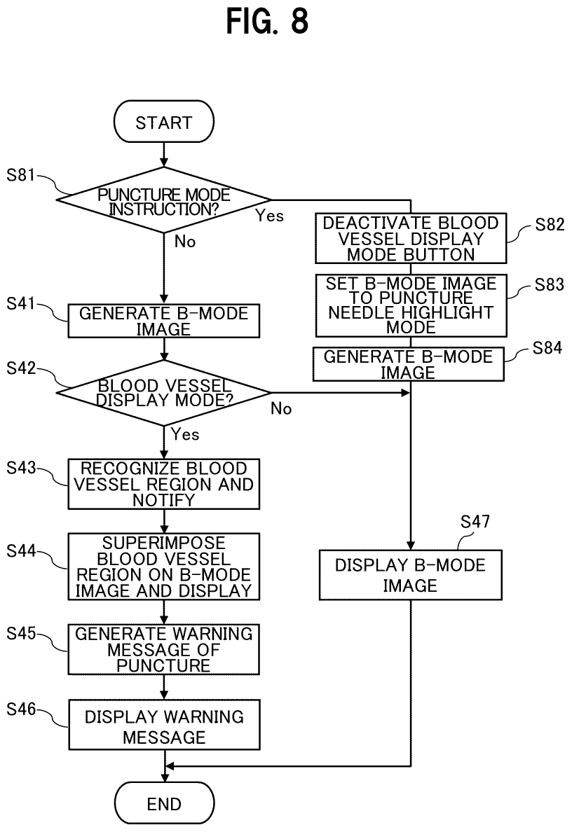

FIG. 8 is a flowchart for explaining the processing of the ultrasound diagnostic apparatus 1 in FIG. 7.

The flowchart of FIG. 8 is obtained by adding the processes of steps S81 to S84 to the flowchart of FIG. 4 for explaining the operation for superimposing and displaying the information indicating the position of the blood vessel region. Here, the processing of the added steps S81 to S84 is described.

In step S81, the controller 9 makes the mode selector 91 detect tapping on the puncturing button 115 instructing to display the puncture mode to determine whether or not the mode is the puncture mode. If the tapping is not the instruction for the puncture mode (No in step S81), the process proceeds to step S41. If the mode is the instruction for the puncture mode (Yes in step S81), the process proceeds to step S82.

In step S82, the controller 9 deactivates the blood vessel button 116 for instructing the blood vessel display mode of the display unit 11 to invalidate the operation instruction of the blood vessel display mode.

In step S83, the controller 9 sets the B-mode image to the puncture needle highlighting mode and proceeds to step S84 to generate a B-mode image.

In step S84, the B-mode image generator 5 generates a B-mode image based on the reflected waves (echoes) of the ultrasound waves, and the process proceeds to step S47. In step S47, the display controller 8 displays the B-mode image on the display unit 11.

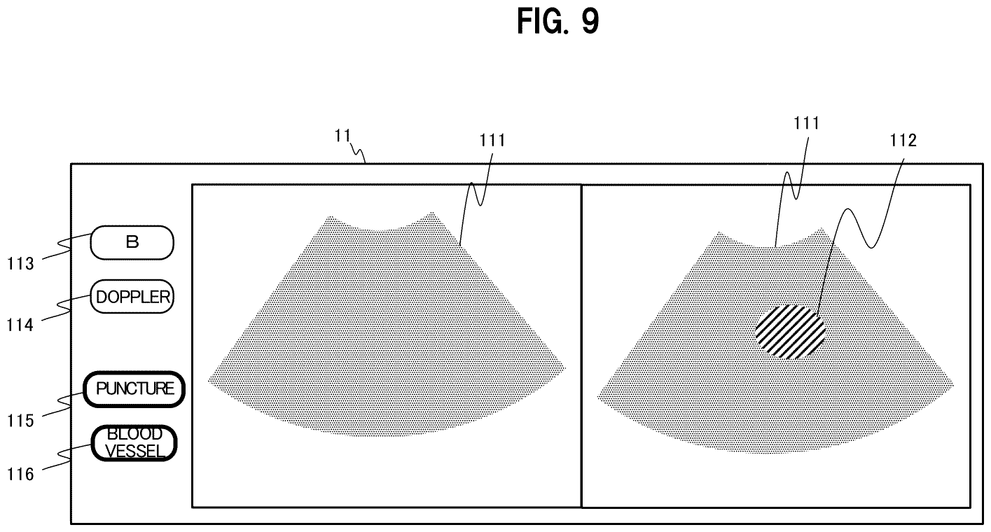

As described above, when the puncture mode is selected, the controller 9 prohibits selection of the blood vessel button 116 instructing the blood vessel display mode for displaying the information indicating the position of a blood vessel region. As a result, because the blood vessel region information 112 is not displayed, the ultrasound diagnostic apparatus 1 can display tissues other than the annotated blood vessel region with high visibility. The above description regards an example in which the ultrasound diagnostic apparatus 1 does not display the information indicating the position of the blood vessel region superimposed on the B-mode image in the puncture mode. Next is a description of another example of handling the information indicating the position of the blood vessel region in the puncture mode with reference to FIG. 9.

FIG. 9 is a diagram illustrating an example in which the ultrasound diagnostic apparatus 1 displays, side by side on the same screen, a B-mode image in which a puncture needle is highlighted and a B-mode image in which the information indicating the position of the blood vessel region is superimposed, when the puncture mode.

This case, unlike FIG. 7, allows even the puncture mode to display the information indicating the position of the blood vessel region allowed only in the blood vessel display mode in FIG. 7.

Specifically, the ultrasound diagnostic apparatus 1, in a state in which only the B-mode image 111 is displayed on the display unit 11, displays the B-mode image 111 of the puncture mode when the user performs tapping on the puncture button 115 for giving an instruction to display the puncture mode. Then, when detecting a tap of the blood vessel button 116 for instructing the blood vessel display mode by the user, the ultrasound diagnostic apparatus 1 displays the B-mode image 111 in which the puncture needle is highlighted and the B-mode image 111 on which the blood vessel region information 112 is superimposed to be displayed, side by side on the same screen. As a result, when the blood vessel region is performed annotation-display using AI, tissues other than the annotated blood vessel region can also be displayed with high visibility.

FIG. 10 is a flowchart for explaining the processing of the ultrasound diagnostic apparatus 1 in FIG. 9.

In step S101, the controller 9 checks whether or not tapping on the puncturing button 115 instructing to display the puncture mode through the mode selector 91 is detected and determines whether or not the mode is in the puncture mode. If the tapping is not the instruction for the puncture mode (No in step S101), the process proceeds to the process of the blood vessel display mode described in FIG. 4. If the tapping is the instruction for the puncture mode (Yes in step S101), the process proceeds to step S102.

In step S102, the controller 9 sets the B-mode image to the puncture needle highlighting mode.

In step S103, the B-mode image generator 5 generates the B-mode image in which the puncture needle is highlighted.

In step S105, the controller 9 determines whether or not the mode is the blood vessels display mode based on the detection of the tapping on the blood vessels button 116 indicating the blood vessels display mode on the touch panel 21 of the operation unit 2. If the mode is the blood vessel display mode (Yes in step S105), the process proceeds to step S106. If the mode is not the blood vessel display mode (No in step S105), the process proceeds to step S109.

In step S106, the blood vessel region recognizer 12 uses the learned model to recognize (determines) a blood vessel region in the B-mode image generated in step S103, and outputs the recognized blood vessel region as information indicating the position of the blood vessel region to the display controller 8.

In step S107, the overlay display controller 81 of the display controller 8 superimposes the information indicating the blood vessel region notified in step S106 on the B-mode image, and displays the superimposed B-mode image on one side of the display unit 11.

In step S108, the display controller 8 displays in parallel the puncture needle highlighted B-mode images generated in step S102 on the other side of the display unit 11, and the processing is ended.

In step S109, the display controller 8 also displays in parallel the puncture needle highlighted B-mode images generated in step S102 on the other side of the display unit 11, and the processing is ended.

The above description specifies the puncture mode as highlighting in the B-mode image the puncture needle inserted with the assistance of the puncture adapter or the puncture guide. Alternatively, the puncture mode may be specified as a puncture guide display mode in which a guide of the puncture needle is displayed on the B-mode image, and the similar processing may be performed. Because the guide display of the puncture needle is not hindered by the information indicating the blood vessel region and is not deteriorated in visibility, tissues other than the annotated blood vessel region can also be displayed with high visibility.

The above description is given of the operation of the display controller 8 during the puncture treatment in the combination of the blood vessel display mode and the puncture mode. The display modes related to blood vessels supported by the ultrasound diagnostic apparatus 1 include the Doppler display mode in which a blood flow signal is detected using the Doppler mode to generate a Doppler image 118 to superimpose and display the Doppler image 118 on the B-mode image 111. Hereinbelow, description is given of operations of the display controller 8 during the puncture treatment in the blood vessel display mode and the Doppler display mode.

Specifically, the Doppler display mode is a technology for detecting a blood flow signal and imaging a blood flow using the Doppler mode (color, power, and the like). The ultrasound diagnostic apparatus 1 displays the velocity of the blood flow using the color Doppler mode and displays the intensity of the blood flow using the power Doppler mode. The user can grasp the state of the blood flow such as the direction, speed, and strength of the blood flow from these Doppler images.

A discrepancy may occur between the blood flow image displayed in the Doppler display mode and the information indicating the position of the blood vessel region displayed in the blood vessel display mode. For this reason, the ultrasound diagnostic apparatus 1 performs the display control described below to allow tissues other than the annotated blood vessel region also to be displayed with high visibility.

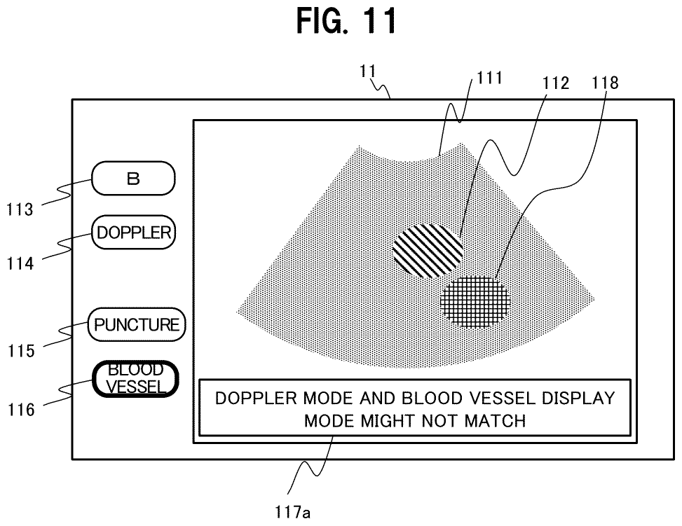

FIG. 11 is a diagram illustrating a display example in a case in which a user taps a blood vessel button 116 for instructing a blood vessel display mode while the ultrasound diagnostic apparatus 1 is displaying a Doppler display mode in which a Doppler image 118 is superimposed and displayed on a B-mode image 111. The Doppler display mode is assumed to be activated in response to a user's tapping on the Doppler button 114 for setting the Doppler display mode.

When the user taps the blood vessel button 116 instructing the blood vessel display mode and the operation unit 2 detects a tapping through the touch panel 21, the ultrasound diagnostic apparatus 1 superimposes and displays the blood vessel region information 112 on the B-mode image 111 on the display unit 11. Furthermore, the ultrasound diagnostic apparatus 1 displays a warning message 117a for preventing puncturing on the display unit 11. The warning regarding the puncture is, for example, “There is a possibility that the Doppler mode and the blood vessel display mode might not match.”

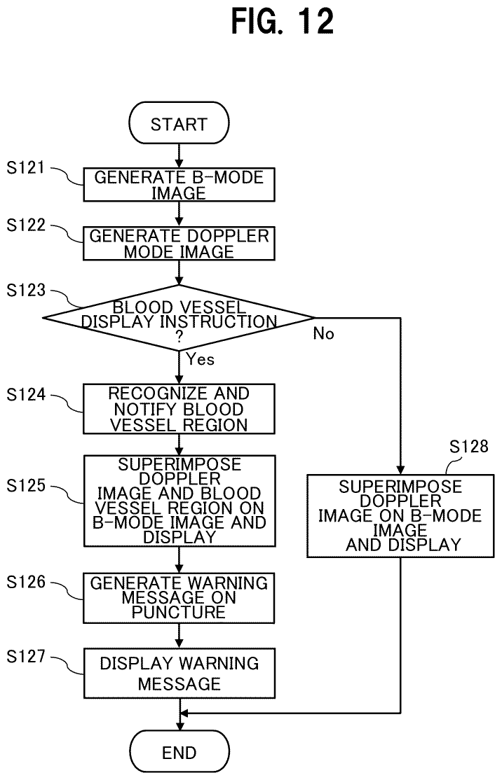

FIG. 12 is a flowchart for explaining the processing of the ultrasound diagnostic apparatus 1 in FIG. 11. The flowchart of FIG. 12 illustrates processing for one frame of a reflected wave (echo) of an ultrasound wave after the Doppler display mode is activated in response to the user's tapping on the Doppler button 114 for setting the Doppler display mode.

In step S121, the B-mode image generator 5 generates data of the B-mode image 111 based on the reflected waves (echoes) of the ultrasound waves.

In step S122, the Doppler mode image generator 7 generates data of a Doppler mode image as the Doppler image 118, based on the reflected waves (echoes) of the ultrasound waves.

In step S123, the controller 9 checks whether or not the mode selector 91 detects tapping on the blood vessels button 116 instructing the blood vessels display mode to determine the presence or absence of an operation instruction for the blood vessels display mode. When there is no operation instruction (No in S123), the process proceeds to step S128, and when there is an operation instruction (Yes in S123), the process proceeds to step S124.

In step S124, the blood vessel region recognizer 12 recognizes (determines) a blood vessel region in the B-mode image generated in step S121 using the learned model, and notifies the display controller 8 of the recognized blood vessel region as the blood vessel region information 112.

In step S125, the overlay display controller 81 of the display controller 8 superimposes the blood vessel region information 112 notified in step S124 and the Doppler mode image (Doppler image 118) generated in step 122 on the B-mode image generated in step S121, and displays the superimposed image on the display unit 11.

In step S126, the warning handler 82 of the display controller 8 generates a warning message related to puncture.

In step S127, the display controller 8 displays the warning message generated in step S126 on the display unit 11, and the processing ends.

In Step S128, the display controller 8 superimposes the B-mode image generated in Step S121 and the Doppler mode image (Doppler image 118) generated in Step S122, displays the superimposed image on the display unit 11, and the processing ends.

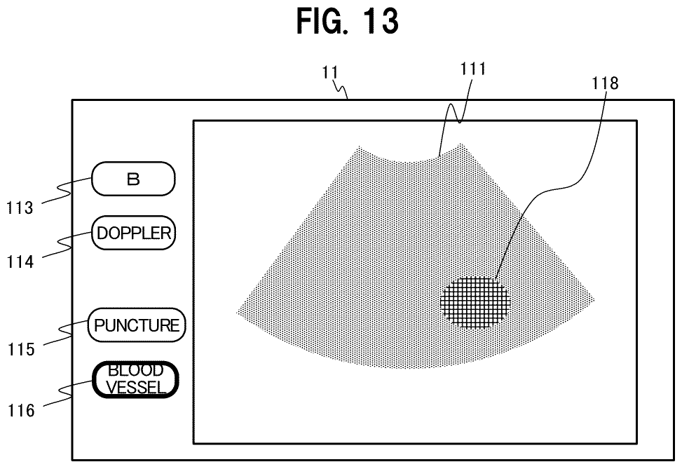

Next, a description is given of another display example in a case in which the user taps the blood vessel button 116 for instructing the blood vessel display mode during the Doppler display mode is displayed with reference to FIG. 13. In FIG. 13, similarly to FIG. 11, the Doppler display mode is assumed to be activated in response to a user's tapping on the Doppler button 114 for setting the Doppler display mode.

Even when the user taps the blood vessel button 116 for instructing the blood vessel display mode and the operation unit 2 of the ultrasound diagnostic apparatus 1 detects the tapping through the touch panel 21, the blood vessel region information 112 is not superimposed to be displayed on the B-mode image 111. Furthermore, the ultrasound diagnostic apparatus 1 does not display the warning message 117 for preventing puncturing. That is, the ultrasound diagnostic apparatus 1 disables the function of the blood vessel display mode in which the blood vessel region information 112 is superimposed to be displayed on the B-mode image 111 while the Doppler display mode is being displayed.

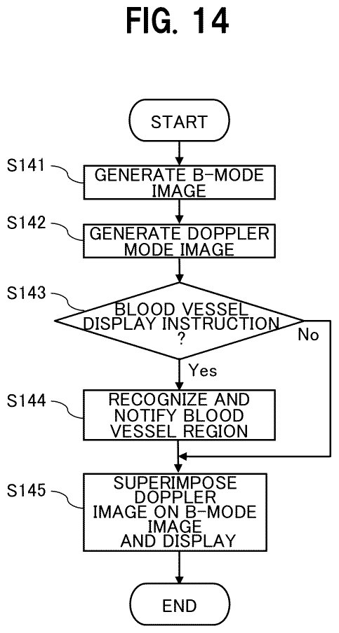

FIG. 14 is a flowchart for explaining the processing of the ultrasound diagnostic apparatus 1 in FIG. 13. The flowchart of FIG. 14 illustrates processing for one frame of a reflected wave (echo) of an ultrasound wave after the Doppler display mode is activated in response to a user's tapping on the Doppler button 114 for setting the Doppler display mode.

In step S141, the B-mode image generator 5 generates the B-mode image 111 based on the reflected waves (echoes) of the ultrasound waves.

In step S142, the Doppler mode image generator 7 generates a Doppler mode image as the Doppler image 118, based on the reflected waves (echoes) of the ultrasound waves.

In step S143, the operation unit 2 detects the tapping of the blood vessel button 116 for instructing the blood vessel display mode by the user through, based on which the controller 9 determines whether or not the tapping is the instruction for the blood vessel display mode. If the tapping is not the instruction for the blood vessel display mode (No in step S143), the process proceeds to step S145. If the tapping is the instruction for the blood vessel display mode (Yes in step S143), the process proceeds to step S144.

In step S144, the blood vessel region recognizer 12 recognizes (determines) a blood vessel region in the B-mode image generated in step S141 using the learned model, and notifies the display controller 8 of the recognized blood vessel region as the blood vessel region information 112.

In step S145, the display controller 8 superimposes the Doppler mode image (Doppler image 118) generated in step S141 on the B-mode image generated in step S142, displays the superimposed image on the display unit 11, and ends the processing.

In the above description, even if the blood vessel region information 112 is obtained in response to the operation instruction for the blood vessel display mode, the information is not superimposed to be displayed on the B-mode image 111 and a warning is not displayed, but the blood vessel button 116 for giving an instruction for the blood vessel display mode may be inactivated so that the user cannot give an operation instruction.

Descriptions with reference to FIG. 11 to FIG. 14 are given of the display control in a case in which there is given an operation instruction for the blood vessel display mode during displaying the Doppler display mode. Next, a description is given of a display control in a case in which there is given an operation instruction for the Doppler display mode during displaying the blood vessel display mode.

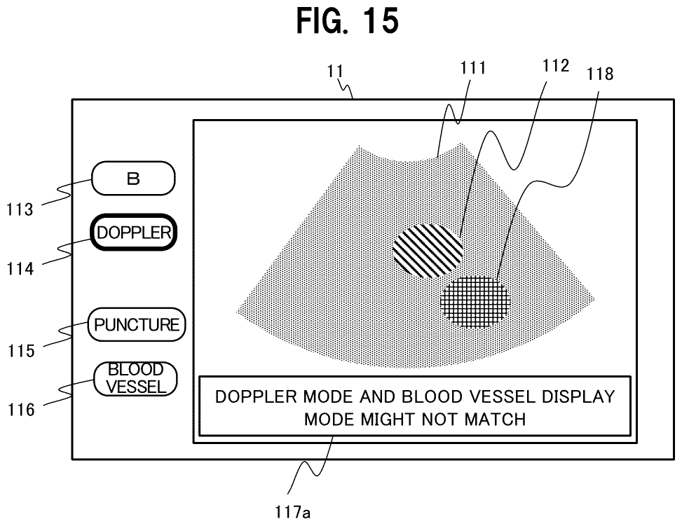

FIG. 15 is a diagram illustrating a display example in a case in which the Doppler button 114 for setting the Doppler display mode is tapped while the ultrasound diagnostic apparatus 1 is displaying the blood vessel display mode in which the blood vessel region information 112 is superimposed and displayed on the B-mode image 111. It is assumed that the blood vessel display mode is activated in response to a tapping on the blood vessel button 116 for instructing the blood vessel display mode.

When the user taps the Doppler button 114 for setting the Doppler display mode and the operation unit 2 detects the tapping through the touch panel 21, the ultrasound diagnostic apparatus 1 displays a display image of ultrasound echoes in which the Doppler image 118 is added to and superimposed on the B-mode image 111. Further, the ultrasound diagnostic apparatus 1 displays a warning message 117a for preventing puncturing, while displaying the display image. The warning regarding the puncture is, for example, “There is a possibility that the Doppler mode and the blood vessel display mode do not match.”

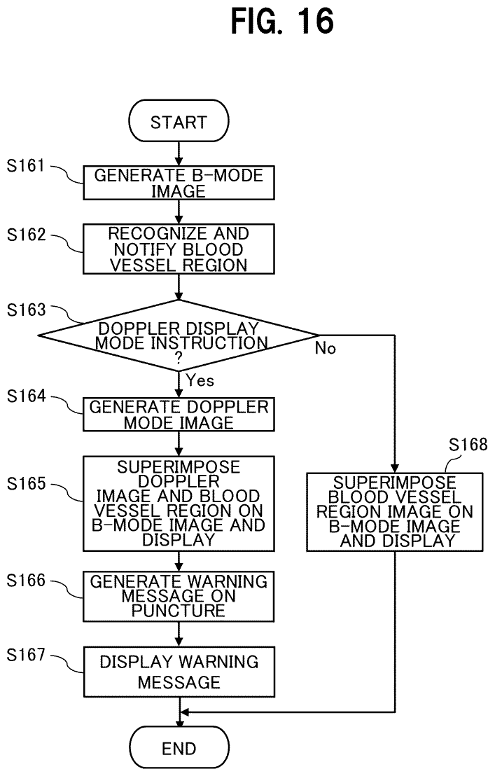

FIG. 16 is a flowchart for explaining the processing of the ultrasound diagnostic apparatus 1 in FIG. 15.

In step S161, the B-mode image generator 5 generates the B-mode image 111 based on the reflected waves (echoes) of the ultrasound waves.

In step S162, the blood vessel region recognizer 12 recognizes (determines) a blood vessel region in the B-mode image generated in step S161 using the learned model, and notifies the display controller 8 of the recognized blood vessel region as the blood vessel region information 112.

In step S163, the controller 9 determines, based on detection of a tapping of the Doppler button 114 for setting the Doppler display mode through the touch panel 21 performed by the operation unit 2, whether or not the tapping is an instruction for the Doppler display mode. If the tapping is not an instruction for the Doppler display mode (NO in step S163), the process advances to step S168. If the tapping is an instruction for the Doppler display mode (YES in step S163), the process advances to step S164.

In step S164, the Doppler mode image generator 7 generates a Doppler mode image as the Doppler image 118, based on the reflected waves (echoes) of the ultrasound waves.

In step S165, the overlay display controller 81 of the display controller 8 superimposes the blood vessel region information 112 notified in step S162 and the Doppler mode image (Doppler image 118) generated in step 164 on the B-mode image generated in step S161 to display the superimposed image on the display unit 11.

In step S166, the warning handler 82 of the display controller 8 generates a puncture-related warning message 117.

In step S167, the display controller 8 displays the warning message 117 generated in step S166 on the display unit 11, and the processing ends.

In Step S168, the display controller 8 superimposes the B-mode image generated in Step S161 and the Doppler mode image (Doppler image 118) generated in Step S164 to display the superimposed image on the display unit 11, and the processing ends.

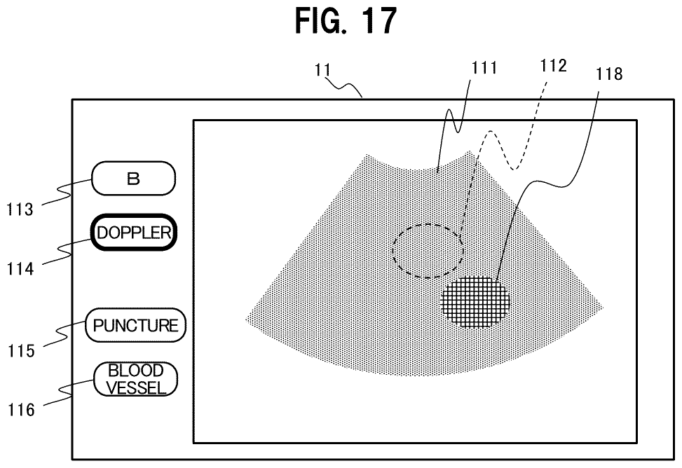

Next, a description is given of another display example in a case in which the user taps the Doppler button 114 for setting the Doppler display mode while the blood vessel display mode is displayed with reference to FIG. 17. In FIG. 17, similarly to FIG. 15, the blood vessel display mode is assumed to be activated in response to a user's tapping on the blood vessel button 116 that instructs the blood vessel display mode.

When the user taps the Doppler button 114 for setting the Doppler display mode, and the operation unit 2 detects the tapping through the touch panel 21, the ultrasound diagnostic apparatus 1 hides the blood vessel region information 112 superimposed on the B-mode image 111. When the warning message related to puncturing is displayed, the ultrasound diagnostic apparatus 1 hides the warning message. Then, the ultrasound diagnostic apparatus 1 displays a display image of an ultrasound echo in which the Doppler image 118 is superimposed on the B-mode image 111. That is, the ultrasound diagnostic apparatus 1 stops the blood vessel display mode, and switches the display to the Doppler display mode.

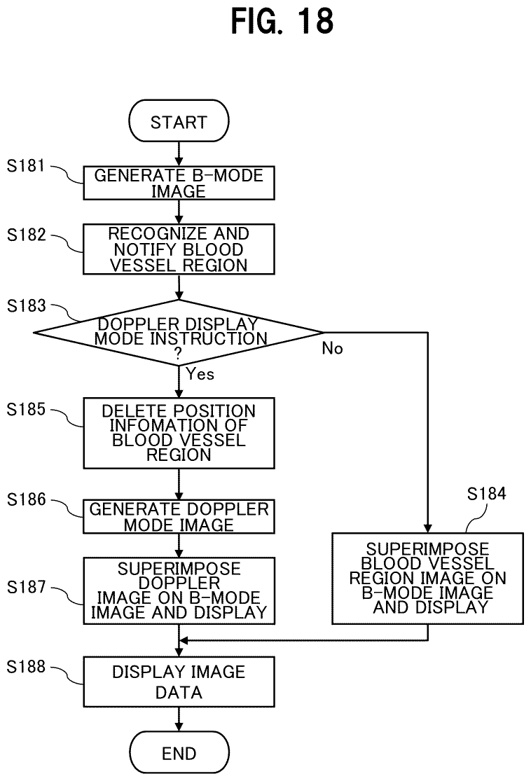

FIG. 18 is a flowchart for explaining the processing of the ultrasound diagnostic apparatus 1 in FIG. 17.

In step S181, the B-mode image generator 5 generates the B-mode image 111 based on the reflected waves (echoes) of the ultrasound waves.

In step S182, the blood vessel region recognizer 12 recognizes (determines) a blood vessel region in the B-mode image generated in step S181 using the learned model, and notifies the display controller 8 of the recognized blood vessel region as the blood vessel region information 112.

In step S183, the display controller 8 determines whether or not a tapping is an instruction for the Doppler display mode based on detection performed by the operation unit 2 of the tapping of the Doppler button 114 for setting the Doppler display mode on the touch panel 21. If the tapping is the instruction for the Doppler display mode (Yes in step S183), the process advances to step S185.

If the instruction is not for the Doppler display mode (No in step S183), the process advances to step S184.

In step S184, the overlay display controller 81 of the display controller 8 superimposes the information indicating the blood vessel region notified in step S182 on the B-mode image generated in step S181 and displays the superimposed image on the display unit 11, and the processing in FIG. 18 ends.

In step S185, the display controller 8 deletes the information indicating the position of the blood vessel region notified in step S182 in order to hide the blood vessel region information 112.

In step S186, the Doppler mode image generator 7 generates a Doppler mode image as the Doppler image 118 based on the reflected waves (echoes) of the ultrasound waves.

In step S187, the overlay display controller 81 of the display controller 8 superimposes the Doppler mode image (the Doppler image 118) generated in step 186 on the B mode image generated in step S181 and displays the superimposed image on the display unit 11, and the processing in FIG. 18 ends. [Effects of the Embodiment]

According to the above-described embodiments, when performing annotation-display of a blood vessel region using an AI technology, a tissue other than the annotated blood vessel region can also be displayed with high visibility.

The present invention is not limited to the above-described embodiments and includes various modifications. The above-described embodiments are described in detail for easy understanding of the present invention, and the present invention is not limited to those having all or parts of the configurations described above. The scope of the invention should be interpreted in terms of the appended claims. Furthermore, a part of a configuration of an embodiment can be replaced with a configuration of another embodiment, and a configuration of an embodiment can be additionally provided with a configuration of another embodiment.
1: Ultrasound Diagnostic Apparatus
2: Operation Unit (Display Instructor)
3: Transmitter
4: Receiver
5: B-Mode Image Generator
6: Roi Setting Unit
7: Doppler Mode Image Generator
8: Display Controller
81: Overlay Display Controller
82: Warning Handler
9: Controller
91: Mode Selector
10: Storage Unit
11: Display Unit
12: Blood Vessel Region Recognizer
100: Main Body
101: Ultrasound Probe

What is claimed is:

1. An ultrasound diagnostic apparatus comprising a processor, the processor configured to execute a prevention function of preventing puncture from being performed in a state in which information indicating a position of a blood vessel region recognized using a learned model is displayed on a display screen.

2. The ultrasound diagnostic apparatus according to claim 1, the processor configured to execute notifying a warning regarding puncture in a case in which the puncture is detected when superimposing on a ultrasound image the information indicating the position of the blood vessel region to display superimposed image on the display screen, wherein the prevention function is implemented by the notifier.

3. The ultrasound diagnostic apparatus according to claim 2, wherein the processor is configured to execute a display control to display a warning about the puncture on the display screen.

4. The ultrasound diagnostic apparatus according to claim 3, wherein the warning is a warning for preventing puncture while displaying the information indicating the position of the blood vessel region superimposed on the ultrasound image.

5. The ultrasound diagnostic apparatus according to claim 3, including a display instructor for displaying the information indicating the position of the blood vessel region superimposed on the ultrasound image, wherein the processor is configured to execute displaying the warning on the display screen when the display instructor is selected.

6. The ultrasound diagnostic apparatus according to claim 2, including a selector for selecting a puncture mode for puncturing; wherein the processor is configured to execute a display control for superimposing the information indicating the position of the blood vessel region on the ultrasound image and selectively displaying the superimposed image on the display screen, wherein the processor is configured to execute the display control to implement the prevention function of the puncture by displaying the ultrasound image in a state in which the information indicating the position of the blood vessel region is not displayed when the puncture mode is selected.

7. The ultrasound diagnostic apparatus according to claim 1, including: a selector for selecting a puncture mode for puncturing, wherein the processor is configured to execute a display control for displaying the information indicating the position of the blood vessel region on the display screen; and the processor is configured to execute a control to receive display specification information specifying information displayed by the display controller, and wherein the processor is configured to implement the prevention function by disabling display of a display instructor for displaying the information indicating the position of the blood vessel region when the puncture mode is selected.

8. The ultrasound diagnostic apparatus according to claim 1, including: a selector for selecting a puncture mode for puncturing, wherein the processor is configured to execute a display control to display the information indicating the position of the blood vessel region on the display screen; and the processor is configured to execute a control to receive display specification information specifying information displayed by the display controller, and wherein the processor is configured to implement the prevention function by rejecting reception of the display instructor for displaying the information indicating the position of the blood vessel region when the puncture mode is selected.

9. The ultrasound diagnostic apparatus according to claim 2, including: a selector for selecting a puncture mode for puncturing; wherein the processor is configured to execute a display control for selectively displaying the information indicating the position of the blood vessel region on the display screen, and wherein the display control implements the prevention function by displaying on the same display screen a B-mode image in which the information indicating the position of the blood vessel region is not displayed and a B-mode image in which the information indicating the position of the blood vessel region is displayed when the puncture mode is selected.

10. The ultrasound diagnostic apparatus according to claim 7, wherein the puncture mode is a puncture needle highlighting mode or a puncture guide display mode.

11. The ultrasound diagnostic apparatus according to claim 3, having a Doppler display mode of detecting a blood flow signal by using a Doppler mode to generate and display a Doppler image, wherein the processor is configured to execute the display control to display the information indicating the position of the blood vessel region and the warning on the display screen, when the Doppler image is displayed on the display screen.

12. The ultrasound diagnostic apparatus according to claim 3, having a Doppler display mode that detects a blood flow signal using a Doppler mode to generate and display a Doppler image, wherein the processor is configured to execute the display control to display nothing of the information indicating the position of the blood vessel region on the display screen and to display nothing of the warning on the display screen when the Doppler image is displayed on the display screen.

13. The ultrasound diagnostic apparatus according to claim 3, having a Doppler display mode that detects a blood flow signal using a Doppler mode to generate and display a Doppler image, wherein the processor is configured to execute the display control to display the information indicating the position of the blood vessel region superimposed on the Doppler image and to display the warning on the display screen in a case of using the Doppler display mode when the information indicating the position of the blood vessel region is being displayed on the display screen.

14. The ultrasound diagnostic apparatus according to claim 3, having a Doppler display mode that detects a blood flow signal using a Doppler mode to generate and display a Doppler image, wherein the processor is configured to execute the display control to stop display of the information indicating the position of the blood vessel region to display the Doppler image and to stop display of the warning on the display screen in a case of using the Doppler display mode when displaying the information indicating the position of the blood vessel region on the display screen.

15. The ultrasound diagnostic apparatus according to claim 1, wherein the processor is configured to execute the display control to stop display of the information indicating the position of the blood vessel region and to display the ultrasound image when a puncture needle is detected in a state in which the information indicating the position of the blood vessel region is displayed on the display screen.

16. A non-transitory computer-readable storage medium storing an ultrasound diagnostic program for causing a computer to execute:
superimposing information indicating a position of a blood vessel region on an ultrasound image and displaying the superimposed image, the position of the blood vessel region being recognized by applying a learned model to the ultrasound image; and
preventing puncture from being performed in a state in which the information indicating the position of the blood vessel region is displayed.

17. An ultrasound diagnostic method executed by a computer, the method comprising:
superimposing information indicating a position of a blood vessel region on an ultrasound image and displaying the superimposed image, the position of the blood vessel region being recognized by applying a learned model to the ultrasound image; and
preventing puncture from being performed in a state in which the information indicating the position of the blood vessel region is displayed.
